SEQUENCE LISTING (2 sequences)

SEQ ID NO: 1 - DNA, 1227 bp, Artificial Sequence
  recombinant polynucleotide
atggagagcggcagccccgagatcgagaagctgagtcagagcgacatctactgggacagcatggtgagcatcaccgagaccggcgtggaggaggtgttcgacctgaccgtgcccggcccccacaacttcgtggccaacgacatcatcgtccacaacagcnnnnnnnnnnnntgcatcagcggcgacagcctgatcagcctggccagcaccggcaagagggtgagcatcaaggacctgctggacgagaaggacttcgagatctgggccatcaacgagcagaccatgaagctagagagcgccaaggtgagcagggtgttctgcaccggcaagaagctagtgtacatcctaagaaccaggctaggcaggaccatcaaggccaccgccaaccacaggttcctaaccatcgacggctggaagaggctagacgagctaagcctaaaggagcacatcgccctaccccggaagctagagagcagcagcctacagctaggcctccgcggccagatcgatgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagcttgagtacaacttcaacagccacaacgtgtatatcatggccgacaagcagaagaacggcatcaaggccaacttcaagatccgccacaacatcgaggacggatccgtgcagctcgccgaccactaccagcagaacaccccaattggcgacgggcccgtgctgctgcccgacaaccactacctgagcacccagagcgctctttcgaaagaccccaacgagaagcgcgatcatatggtcctgctcgagttcgtgaccgccgccgggatcactctcggcatggacgagctgtacaagtaa

SEQ ID NO: 2 - protein, 408 aa, Artificial Sequence
  VARIANT 54, 55, 56, 57: Xaa = Any Amino Acid
MESGSPEIEKLSQSDIYWDSMVSITETGVEEVFDLTVPGPHNFVANDIIVHNSXXXXCISGDSLISLASTGKRVSIKDLLDEKDFEIWAINEQTMKLESAKVSRVFCTGKKLVYILRTRLGRTIKATANHRFLTIDGWKRLDELSLKEHIALPRKLESSSLQLGLRGQIDVSKGEELFTGVVPILVELDGDVNGHKFSVSGEGEGDATYGKLTLKFICTTGKLPVPWPTLVTTLTHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2006/0275832 A1
(43) Pub. Date: Dec. 7, 2006

(54) Methods of screening cyclic peptides and identifying targets therefor

(76) Inventor: Esteban Masuda, Menlo Park, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/533,144

(22) PCT Filed: Aug. 30, 2003
(86) PCT No.: PCT/US03/27370

§ 371 Date: Apr. 24, 2006

Related U.S. Application Data
(60) Provisional application No. 60/407,385, filed on Aug. 30, 2002.

(51) Int. Cl. G01N 33/567; A61K 38/12

(52) U.S. Cl. 435/7.2; 514/9

(57) ABSTRACT

The present invention provides compositions and methods for identifying candidate compounds capable of altering a phenotype of a cell that mediate their activity by binding with a specific chaperone of interest. Also provided are methods and compositions for identifying the target of the candidate compound-chaperone complex.

### CROSS REFERENCES TO RELATED APPLICATIONS

This application claims the benefit of U.S. Provisional Application No. 60/407,385, filed Aug. 30, 2002, the entire contents of which is incorporated herein by reference.

### FIELD OF THE INVENTION

The present invention relates to improved methods of screening libraries of cyclic peptides that utilizes a chaperone molecule capable of binding a portion or region of the cyclic peptide library members. Use of the chaperone permits the identification of cyclic peptides that alter a phenotype of a cell when complexed with the chaperone, as well as isolation and identification of the target protein with which the complex interacts.

### BACKGROUND OF THE INVENTION

The recent advent of myriad library screening methodologies permits the rapid screening of libraries of thousands, and in many instances, even millions or trillions, of compounds to identify those compounds within the library having a desired property, such as for example, the ability to bind a particular target protein of interest or the ability to agonize or antagonize a cellular function of interest. These latter functional screening assays are of particular importance, as they permit the identification of compounds capable of effecting a certain biological response, such as the ability to alter a phenotype of a cell in a particular fashion, without knowledge of the target proteins responsible for the response. One such functional assay utilizes cell lines (typically human) stably transfected with a construct comprising a reporter gene operably linked to an element, such as a promoter, responsive to a specific effector molecule of interest. Agonists or antagonists of the specific effector molecule, or a molecule in a complex signal transduction pathway including the effector molecule, can be readily identified by administering a candidate compound to the stably transfected cells and monitoring the expression, either at the transcriptional or translational level, of the reporter gene. Other functional assays bypass the need to create such constructs by monitoring cells comprising candidate compounds for detectable or observable changes in phenotype, which can range from a change in cellular morphology, viability, growth, etc. to changes in the expression of one or more endogenous gene products, to changes in the ability of the cell to transmit signals intracellularly or intercellularly to other cells.

Screening molecular libraries of compounds for the ability to alter a specific phenotype of a cell, such as the ability to modulate intra- or intercellular signal transduction cascades or pathways, has led to discoveries of significant clinical importance. For example, both cyclosporin A (CsA) and FK506, which are potent immunosuppressants administered to organ transplant recipient to prevent organ rejection, were identified in pharmaceutical screening assays designed to detect inhibition of T-cell activation. Although CsA and FK506 bind different target proteins—cyclophilin A (CypA) and FKM06 binding protein (FKBP), respectively—the biological effect of both compounds is virtually the same: profound and specific suppression of T-cell activation, phenotypically observable in T-cells as inhibition of mRNA production dependent upon transcription factors such as NF-AT and NF-κB.

One class of molecules that has been shown to play an important role in modulating signaling pathways is peptides. The literature is replete with examples of peptides capable of modulating such pathways. For example, a peptide derived from the HIV-1 envelope protein has been shown to block the action of cellular calmodulin. Peptides corresponding to regions of the Fas cytoplasmic domain have been shown to have death-inducing or G protein inducing functions. Peptides corresponding to the protein kinase C isozyme βPKC block nuclear translocation of βPKC in Xenopus oocytes following stimulation. Moreover, the two immunosuppressants discussed above, CsA and FK506, although not composed entirely of genetically encoded amino acids, are cyclic peptides.

These few examples highlight the importance of peptides, both linear and cyclic, as candidate compounds in pharmaceutical screening assays. Cyclic peptides are particularly attractive candidate compounds because they generally have greater bioavailability and are more resistant to in vivo degradation by enzymes such as proteases than their linear counterparts. To this end, several methodologies suitable for delivering and screening libraries of linear and cyclic peptides for functional activity have been developed. Two exemplary assay systems are described in U.S. Pat. No. 6,153,380 and WO 01/66565. Both of these exemplary systems utilize retroviral vectors to deliver libraries of polynucleotides to target cells to screen for and identify expression products of the polynucleotides that alter a phenotype of the target cells. The system described in WO 01/66565 is specifically adapted to screening libraries of expressed cyclic peptides.

Although powerful, assay systems designed to screen cyclic peptides are not without drawbacks. Once a candidate compound having a desired biological property of interest is identified, it is often desirable to identify the target molecule(s) with which the identified compound interacts. For linear peptides this is typically accomplished using yeast two-hybrid assays or variants of such assays. However, cyclic peptides are not amenable to yeast two-hybrid assays. As a consequence, identifying targets that interact with biologically active cyclic peptides provides significant challenges.

Random functional screening assays provide significant challenges, as well. It is well established that intracellular signaling occurs through a series of protein-protein interactions. In many instances, candidate compounds such as linear and peptides and cyclic peptides may not have enough size or bulk to disrupt or effect such protein-protein interactions. In fact, the art is replete with examples of small molecules, for example cyclic peptides, that exert their biological effect when complexed with larger “chaperone” proteins. Presumably, the specificity of the biological effect is provided by the small molecule and the chaperone protein provides bulk and a larger interaction surface than that provided by the small molecule alone. Specific examples of small molecules that exert their biological activity when complexed with larger chaperone proteins are the immunosuppressant drugs cyclosporin A (CsA), FK506 and Rapamycin (“Rap”). The immunosuppressant activity of both CsA and FK506 derives from the formation of a complex with their respective immunophilins (CypA and FKBP, respectively). The CsA-CypA and FK506-FKBP complexes inhibit or interfere with cytoplasmic signaling cascades leading to transcription of lymphokine genes in T-cells (Schreiber, 1991, Science 251:283-287; Bierer et al, 1991 Proc. Natl. Acad. Sci. USA 87:9231-9235; Dumont et al., 1990, J. Immunol. 144:251-258), presumably by binding to, and inhibiting the activity of, the phosphatase calcineurin (see, e.g., Cameron, et al., 1997, J. Biol. Chem. 272:27582-27588). Similarly, a complex formed between FKBP and Rap, which is structurally related to FK506, inhibits growth factor-dependent signaling cascades that, if not so inhibited, lead to proliferation of T-cells (Schreiber, 1992, Tetrahedron 48:2545-2558). The Rap-FKBP complex has also been shown to inhibit the phosphorylation of a 70 kD S6 protein linase, leading a decrease in protein synthesis accompanying cell cycle progression (Calvo et al., 1992, Proc. Natl. Acad. Sci. USA 89:7571-7575; Chung et al., 1992, Cell 69:1227-1236; Kuo et al., 1992, Nature 358:70-73; Price et al., 1992, Science 257:973-977). The dephosphorylation of p70S^{6K} is believed to be mediated by the formation of a ternary complex of FKBP12, Rap and FKBP12-rapamycin-associated protein (FRAP) (Desai, et al., 2001, Proc. Natl. Acad. Sci. 99:4319-4324). The Rap-FKBP complex also inhibits and to inhibit activation of cdk2/cyclin E complex (Flanagan et al., 1993, J. Cell Biochem. 17A:292). In all of these cases, the activities of CsA, FFK506 and Rap are not mediated by direct binding to the effector molecule, but by action of a CypA-, FK506- or Rap-immunophilin complex on upstream component(s) which regulate the effector molecule.

Quite interestingly, the biological effects of FK506-FKBP and RaP-FKBP complexes are distinct. FKBP12 has been shown to bind a specific lencine-proline sequence (1400-1401) of the inositol 1,4,5-triphosphate receptor (IP3R) and anchors calcincurin to this domain (see, e.g., Cameron et al., 1997, J. Biol. Chem. 272:27582-27588). The Leu-Pro structure resembles a portion of the structure common to both FK506 and Rap. The similarity in structure between the FKBP12 binding region of IB3R, FK506 and Rap suggests that this structure is essential to the binding of EK506 and Rap to FKBP12. The differences in the biological activities FK506 and Rap suggest that the remainder of the molecules hold the key to their specificities. Presumably, the FKBP, and CyPA in the case of CsA, act as “chaperones” that “present” the CsA, FK506 or Rap to the targets with which the respective complexes interact.

It would be highly desirable to have available screening methods that are able to take advantage of the ability of such “chaperone” proteins, such as immunophilins, to “present” small candidate compounds to target molecules to effect or disrupt protein-protein interactions in order to identify candidate compounds having specified biological activities, such as the ability to alter a phenotype of a cell. Moreover, it would be highly desirable to have available screening methods which aid the identification of the target proteins with which the identified candidate compound interacts to exert its biological effect(s).

### SUMMARY OF THE INVENTION

These and other objects are furmished by the present invention, which provides methods and compositions for screening candidate compounds, such as peptides and cyclic peptides, that take advantage of the ability of certain proteins to act as “chaperones” to “present” biologically active molecules such as cyclic peptides to target molecules. The methods provide for the identification of candidate compounds that alter a phenotype of a target cell when complexed with a specified chaperone protein, and also provide a means for isolating and identifying the target protein with which the candidate compound-chaperone complex interacts. In preferred embodiments, the methods and compositions provide for the identification of candidate compounds that modulate, and in particular inhibit, intra- and/or extracellular signal transduction cascades when complexed with immunophilias such as FK binding proteins and cyclophilins.

Accordingly, in one aspect, the present invention provides a method of identifying compounds that are capable of altering a phenotype of a cell by binding or interacting with a specified chaperone protein. The method generally comprises administering to a cell a candidate compound of interest that comprises two regions or domains: a chaperone binding region and a putative target binding region. The structure of the chaperone binding region is known and is designed to bind a particular chaperone of interest. The structure of the putative target binding region is typically unknown, either in whole or in part, and constitutes the portion of the candidate compound being assessed for potential biological activity. The cell is then assessed to determine whether a phenotype of the cell has been altered. Cells comprising active candidate compounds can be analyzed to determine the identity of the active candidate compound, and in particular the structure of its putative target binding region.

In one embodiment of the method, the candidate compound is a cyclic peptide in which one end of the chaperone binding region is fused, either directly or by way of an optional linker, to one end of the putative target binding region and the other end of the chaperone binding region is fused, either directly or by way of an optional linker, to the other end of the putative binding region. The amino acid sequence of the putative target binding region may be completely random, or it may include one or more sub regions of specified sequence(s). In another embodiment of the method, the chaperone binding region of the candidate compound is capable of binding an immunophilin, such as, for example an FK binding protein or a cyclophilin.

In embodiments of the method that employ synthetic candidate compounds that are capable of traversing cell membranes, the candidate compounds may be administered to the cell by contacting the cell with the candidate compound. In embodiments of the method that employ candidate compounds that can be expressed or transcribed by polynucleotides, such as peptides or RNAs, the candidate compound can be administered to the cell via a polynucleotide capable of expressing or transcribing the candidate compound in the cell. In such embodiments, the sequences of active candidate compounds, and in particular the sequences of their respective putative target binding regions, can be determined by sequencing the polynucleotides of cells exhibiting an altered phenotype.

In another aspect, the present invention provides a method of identifying a target molecule with which an identified candidate compound-chaperone complex interacts. The method generally comprises administering to a cell a candidate compound which comprises two regions: a chaperone binding region and a putative target binding region, as previously described. The cell is then assessed for the presence or absence of an altered phenotype, as previously described. A cell lysate of a cell having an altered phenotype is contacted with an agent, which is preferably immobilized on a solid support, which specifically binds the chaperone. Any target protein bound to the chaperone is then isolated and characterized.

In yet another aspect, the present invention provides libraries of candidate compounds, each of which includes chaperone binding region of known structure and a putative binding region of unknown structure (either in whole or in part), polynucleotide libraries capable of expressing, either at the transcription or translation level, RNA or peptide embodiments of such candidate compounds, retroviral libraries comprising the polynucleotide libraries and cellular libraries comprising the retroviral libraries. In one embodiment of the various libraries, the chaperone binding region of each candidate compound is the same. In another embodiment of the various libraries, the putative binding region of each candidate compound is distinct.

The methods and compositions of the invention provide myriad advantages over traditional screening techniques. The complexes formed between the candidate compounds and the chaperone protein are larger than the candidate compound alone, thus providing bulk upon binding that may inhibit the activity of the target via steric hindrance, even in instances where the candidate compound does not bind directly to an active site. Moreover, in instances where the chaperone protein is highly expressed, such as when the chaperone is an immunophilin, the identified candidate compounds will make ideal leads for pharmaceutical development. Coadministration of the chaperone should not be necessary—owing to its high expression, the active complex will form in situ following administration of the identified candidate compound. Moreover, since only a fraction of the chaperone will be occupied by the cyclic peptide, it will be free to carry out its other biological functions.

The methods and compositions of the invention also provide an easy, efficient means of identifying the target with which the complex interacts. Since the chaperone is known, active target-candidate compound-chaperone complexes can be isolated using affinity reagents specific for the chaperone and the target dissociated there from and characterized.

### BRIEF DESCRIPTION OF THE FIGURES

FIG. 1 provides a cartoon illustrating the principles of the invention;

FIG. 2 provides a cartoon illustrating the protein splicing reaction mediated by a contiguous intein;

FIG. 3 provides a cartoon illustrating the presumed mechanism of action of the protein splicing reaction of FIG. 2;

FIG. 4 provides a cartoon illustrating the protein splicing reaction mediated by a split intein to generate a candidate cyclic peptide;

FIG. 5 provides a cartoon illustrating the presumed mechanism of action of the protein splicing reaction of FIG. 4;

FIG. 6 provides a cartoon illustrating a candidate polynucleotide intein construct capable of expressing a candidate cyclic peptide; and

FIG. 7 provides the nucleotide sequence (coding strand) and translated amino acid sequence of the I_{C}-extein-I_{N}-BFP region of the retroviral vector of FIG. 6 (stop codons are indicated with “.”).

### DETAILED DESCRIPTION OF THE PREFERRED EMBODIMENTS

Abbreviations

The abbreviations used for the genetically encoded amino acids are conventional and are as follows:

| Amino Acid | Three-Letter | One-Letter |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L or D-configtiraton about a-carbon (Cu). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the a-carbon and lower case letters designate amino acids in the D-configuration about the α-carbon. For example, “A” designates L-alanine and “a” designates D-alanine. When peptide sequences are presented as a string of one-letter or three-letter abbreviations (or mixtures thereof), the sequences are presented in the N→C direction in accordance with common convention.

The abbreviations used for the genetically encoding nucleo-sdcs are conventional and are as follows: adenosine (A); guanosine (G); cytidine (C); thymidine (T); and uridine (U). Unless specifically delineated, the abbreviated nucleotides may be either ribonucleosides or 2′-deoxyribonucleosides. The nucleosides may be specified as being either ribonucleosides or 2′-deoxyribonucleosides on an individual basis or on an aggregate basis. When specified on an individual basis, the one-letter abbreviation is preceded by either a “d” or an “r,” where “d” indicates the nucleoside is a 2′-deoxyribonucleoside and “r” indicates the nucleoside is a ribonucleoside. For example, “dA” designates 2′-deoxyriboadenosine and “rA” designates riboadenosine. When specified on an aggregate basis, the particular nucleic acid or polynucleotide is identified as being either an RNA molecule or a DNA molecule. Nucleotides are abbreviated by adding a “p” to represent each phosphate, as well as whether the phosphates are attached to the 3′-position or the 5′-position of the sugar. Thus, 5′-nucleotides are abbreviated as “pN” and 3′-nucleotides are abbreviated as “Np,” where “N” represents A, G, C, T or U. When nucleic acid sequences are presented as a string of one-letter abbreviations, the sequences are presented in the 5′→3′ direction in accordance with common convention, and the phosphates are not indicated. cl The Preferred Embodiments

As discussed in the Background section, many intracellular and intercellular processes, such as intra- and intercellular signaling, occur through a series of protein-protein interactions. Oftentimes, such protein-protein interactions are mediated via proteins or molecules that act as “chaperones” for smaller molecules such as cyclic peptides. Specific examples of such chaperone molecules include immunophilins such as FK binding proteins and cyclophilins, which bind cyclic peptides such as CsA, FK506 and Rap to form binary complexes which then interact with and modulate the activity(ies) of other biological molecules or complexes. The present invention provides compositions and methods that capitalize on the activities of such chaperone molecules to screen for and identify candidate compounds that alter a phenotype of a cell, such as, for example, by altering an intracellular or intercellular signaling pathway or cascade when bound to the chaperone molecule. Little or no knowledge of the pathway is required. The only requirement is that the biological process being investigated produce an observable physiologic or phenotypic change in the target cell. The invention also provides for the isolation of the constituents of the pathway, and in particular the target molecules with which the candidate compound-chaperone complex interacts to mediate its activity, the tools to characterize the pathway, and lead compounds for pharmaceutical development.

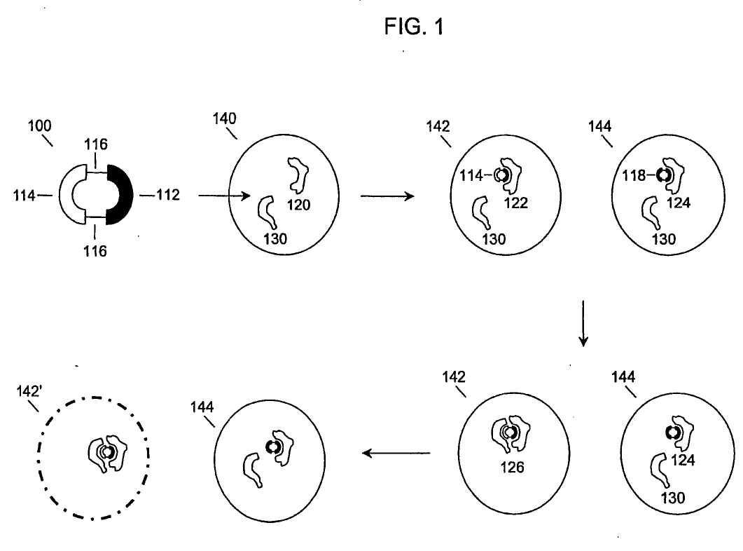

The principles of the methods of the invention are illustrated in FIG. 1. Referring to FIG. 1, a candidate compound 100, which in this case is a cyclic peptide, is administered to cells 140 expressing a chaperone 120, which in preferred embodiments is a highly expressed protein such as an immunophilin (discussed in more detail, below), to yield cell 142. The cells also express numerous putative target proteins, exemplified by effector molecule 130, the activity of which can be modulated by interacting with a complex including chaperone 120. As a specific example, effector molecule 130 is a protein involved in an intracellular signaling cascade. Candidate cyclic peptide 100 comprises two regions: a chaperone binding region 112 and a putative target binding region 114. The chaperone binding region includes a sequence known to interact with chaperone 120 (described in more detail, below). The putative target binding region 114 is typically a sequence that is random or semi-random, as will be discussed in more detail below. The chaperone binding and putative target binding regions may be fused together directly or, as illustrated in FIG. 1, may be spaced apart from one another via linkers 116, which may be the same or different. A different candidate cyclic peptide (not shown), which includes the same chaperone binding region as candidate cyclic peptide 100 but has a different putative target binding region, is administered to cells 140 to yield cell 144. As illustrated, once inside cells 142 and 144, the candidate cyclic peptides bind chaperone 120 via their respective chaperone binding regions to form binary candidate cyclic peptide-chaperone (“CCP-C”) complexes 122 and 124, respectively. When bound to chaperone 120, their respective different putative target binding regions, 114 and 118, are free to interact with other molecules in the cell, such as effector molecule 130. As illustrated, CCP-C complex 122 binds putative target 130 to yield a ternary complex 126. When bound by CCP-C complex 122, the biological activity of effector molecule 130 is modulated, leading to an observable phenotypic change in cell 142 (illustrated as cell 142′). However, CCP-C complex 124 does not bind effector molecule 130, leaving it to carry out its normal biological function(s). As a consequence, cell 144 does not exhibit a change in phenotype.

As exemplified by FIG. 1, by varying the structure or sequence of the putative binding region of the candidate cyclic peptide, cyclic peptides which modulate biological processes can be rapidly identified. Significantly, such cyclic peptides can be identified without any prior knowledge about the effector molecule. Moreover, since the complex formed between the chaperone anid cyclic peptide provides more bulk than the cyclic peptide alone, the present methods are capable of identifying active cyclic peptides that may be missed in traditional screening methods that do not utilize chaperones. When used in a library format to screen a library of candidate cyclic peptides, each of which includes a unique putative target binding region, the methods of the invention provide a powerful means of identifyig cyclic peptides capable of modulating biological processes, many of which may have been missed using traditional screening techniques.

Moreover, the present methods also permit isolation and identification of the target of the CCP-C complex (e.g., effector molecule 130 of FIG. 1). Since the chaperone to which the candidate cyclic peptides bind is known, cells exhibiting a change of phenotype (e.g., cell 142′ of FIG. 1) can be lysed, the ternary complex 126 isolated from the lysate using, for example, affinity reagents that specifically bind chaperone 120. Any target molecule (e.g., effector molecule 130 of FIG. 1) bound to the CCP-C complex 126 can be dissociated from the ternary complex and characterized. As a specific example, ternary complex 126 can be isolated from a lysate of cells 142′ using immunoaffnity chromatography employing an antibody that specifically binds chaperone 120.

A key feature of the methods of the invention is the use of a chaperone that effectively increases the size of the candidate cyclic peptide and also provides a greater number of points of contact for effecting protein-protein interactions with target molecules than the cyclic peptide alone. For example, as evidenced by complex 122 of FIG. 1, this complex has a greater surface area for interacting with target proteins than cyclic peptide 100 alone. As used herein, a “chaperone” may be any molecule or protein capable of “presenting” a cyclic peptide to another molecule. Thus, “chaperones” are intended to include those proteins commonly known in the art as chaperones, as well as any other proteins or molecules capable of binding a candidate cyclic peptide and presenting it to another molecule, as described herein. Typically, such a chaperone molecule will be a protein that is known to, or is suspected of, being involved in or mediating protein-protein interactions. Numerous proteins of this type are known in the art, and include by way of example and not limitation, classical chaperone proteins (e.g., heat-shock proteins), immunophilins, proteases and other enzymes, etc. Specific examples of proteins useful as chaperones in the present methods include, but are not limited to:

Protein Name

| | |
|---|---|
| FK506 binding protein10 | gi\|21361895\|ref\|NP_068758.2\|[21361895] |
| heat shock 70 kD protein binding | gi\|19923193\|ref\|NP_003923.2\|[19923193] |
| protein; progesterone receptor- | |
| associated p48 | |
| protein; putative tumor suppressor | |
| ST13; Hsp70-interacting protein | |
| peptidylprolyl isomerase-like protein | gi\|19557640\|ref\|NP_572028.1\|[19557640] |
| 3 isoform PPIL3b; cyclophilin-like | |
| protein | |
| 3; peptidylprolyl cis-trans | |
| isomerase-like protein 3; PPIase-like | |
| protein 3 | |
| peptidylprolyl isomerase-like protein | gi\|19557636\|ref\|NP_570981.1\|[19557636] |
| 3 isoform PPIL3b; cyclophilin-like | |
| protein | |
| 3; peptidylprolyl cis-trans | |
| isomerase-like protein 3; PPIase-like | |
| protein 3 | |
| FLJ32150 protein; novel protein | gi\|18677724\|ref\|NP_443202.2\|[18677724] |
| similar to KIAA0417 | |
| peptidylprolyl isomerase-like protein | gi\|14277126\|ref\|NP_115861.1\|[14277126] |
| 3 isoform PPIL3a; cyclophilin-like | |
| protein | |
| 3; peptidylprolyl cis-trans | |
| isomerase-like protein 3; PPIase-like | |
| protein 3 | |
| heat shock 70 kD protein 2; Heat- | gi\|13676857\|ref\|NP_068814.2\|[13676857] |
| shock 70 kD protein-2 | |
| stress 70 protein chaperone, | gi\|5902126\|ref\|NP_008879.1\|[5902126] |
| microsome-associated, 60 kD; Stress | |
| 70 protein | |
| chaperone, microsome-associated, | |
| p60; human microsomal stress 70 | |
| protein ATPase | |
| core | |
| heat shock 70 kD protein 6 | gi\|4504515\|ref\|NP_002146.1\|[4504515] |
| (HSP70B′); Heat-shock 70 kD | |
| protein-6 (HSP70B′) | |
| 150 kDa oxygen-regulated protein | gi\|22063141\|ref\|XP_006464.8\|[22063141] |
| precursor (Orp150) | |
| Heat shock cognate 71 kDa protein | gi]22061946\|ref\|XP_171626.1\|[22061946] |
| heat shock 70 kD protein 1-like | gi\|22056764\|ref\|XP_175177.1\|[22056764] |
| heat shock 70 kD protein 8; Heat | gi\|22055166\|ref\|XP_066483.3\|[22055166] |
| shock cognate protein 70 | |
| heat shock protein | gi\|22055141\|ref\|XP_175247.1\|[22055141] |
| heat shock protein | gi\|20555572\|ref\|XP_166348.1\|[20555572] |
| heat shock 70 kD protein 8; Heat | gi\|20550194\|ref\|XP_090256.4\|[20550194] |
| shock cognate protein 70 | |
| HEAT SHOCK 70 KDA PROTEIN | gi\|20547107\|ref\|XP_114482.1\|[20547107] |
| 4 (HEAT SHOCK 70-RELATED | |
| PROTEIN APG-2) | |
| (HSP70RY) | |
| heat shock 70 kD protein 8; Heat | gi\|20540302\|ref\|XP_166540.1\|[20540302] |
| shock cognate protein 70 | |
| heat shock 70 kD protein 8; Heat | gi\|18595470\|ref\|XP_088650.1\|[18595470] |
| shock cognate protein 70 | |
| Hypothetical protein KIAA0417 | gi\|18576822\|ref\|XP_048898.3\|[18576822] |
| 65 kDa Yes-associated protein | gi\|18562439\|ref\|XP_094606.1\|[18562439] |
| (YAP65) | |
| Heat shock cognate 71 kDa protein | gi\|22048365\|ref\|XP_068577.2\|[22048365] |
| HEAT SHOCK 70 KD PROTEIN 6 | gi\|22045701\|ref\|XP_084070.5\|[22045701] |
| (HEAT SHOCK 70 KD PROTEIN | |
| B) | |
| heat shock 70 kD protein 8; Heat | gi\|22043849\|ref\|XP_067703.5\|[22043849] |
| shock cognate protein 70 | |
| protease; reverse transcriptase; | gi\|22043778\|ref\|XP_065747.4\|[22043778] |
| RNaseH; integrase; dUTPase; | |
| Pro-Pol-dUTPase polyprotein | |
| heat shock 70 kD protein 8; Heat | gi\|22042705\|ref\|XP_171283.1\|[22042705] |
| shock cognate protein 70 | |
| heat shock protein | gi\|20542648\|ref\|XP_095553.4\|[20542648] |
| Heat shock cognate 71 kDa protein | gi\|20535117\|ref\|XP_065611.2\|[20535117] |
| 78 kDa glucose-regulated protein | gi\|18546205\|ref\|XP_088941.1\|[18546205] |
| precursor (GRP 78) | |
| (Immunoglobulin | |
| heavy chain binding protein) (BIP) | |
| (Endoplasmic reticulum lumenal | |
| Ca2+ binding | |
| protein grp78) | |
| glycogenin glucosyltransferase | gi\|17437880\|ref\|XP_060491.1\|[17437880] |
| (EC 2.4.1.186) - human | |
| BAG-family molecular chaperone | gi\|12643895\|sp\|Q9UL15\|BAG5_HUMAN[12643895] |
| regulator-5 (BAG-5) | |
| BAG-family molecular chaperone | gi\|12643665\|sp\|O95817\|BAG3_HUMAN[12643665] |
| regulator-3 (BCL-2 binding | |
| athanogene-3) (BAG-3) | |
| (Bcl-2-binding protein Bis) | |
| (Docking protein CAIR-1) | |
| BAG-family molecular chaperone | gi\|12643661\|sp\|O95429\|BAG4_HUMAN[12643661] |
| regulator-4 (Silencer of death | |
| domains) | |
| Telomerase-binding protein p23 | gi\|8928247\|sp\|Q15185\|TEBP_HUMAN[8928247] |
| (Hsp90 co-chaperone) (Progesterone | |
| receptor | |
| complex p23) | |
| Heat shock-related 70 kDa protein 2 | gi\|1708307\|sp\|P54652\|HS72_HUMAN[1708307] |
| (Heat shock 70 kDa protein 2) | |
| Heat shock 70 kDa protein 1 | gi\|462325\|sp\|P08107\|HS71_HUMAN[462325] |
| (HSP70.1) (HSP70-1/HSP70-2) | |
| stress 70 protein chaperone, | gi\|9507149\|ref\|NP_062144.1\|[9507149] |
| microsome-associated, 60 kD human | |
| homolog | |
| nucleophosmin/nucleoplasmin 3 | gi\|6857818\|ref\|NP_008924.1\|[6857818] |
| Heat shock protein 75 kDa, | gi\|17865679\|sp\|Q12931\|TRAL_HUMAN[17865679] |
| mitochondrial precursor (HSP 75) | |
| (Tumor necrosis | |
| factor type 1 receptor associated | |
| protein) (TRAP-1) (TNFR- | |
| associated protein 1) | |
| Nucleoplasmin 3 | gi\|6647592\|sp\|O75607\|NPM3_HUMAN[6647592] |
| BAG-family molecular chaperone | gi\|12644477\|sp\|Q99933\|BAG1_HUMAN[12644477] |
| regulator-1 (BCL-2 binding | |
| athanogene-1) (BAG-1) | |
| (Glucocorticoid receptor-associated | |
| protein RAP46) | |
| importin 4 | gi\|18874099\|ref\|NP_078934.2\|[18874099] |
| importin 4 | gi\|18700635\|gb\|AAL78660.1\|AF411122_1[18700635] |
| Calnexin precursor (Major | gi\|543920\|sp\|P27824\|CALX_HUMAN[543920] |
| histocompatibility complex class I | |
| antigen-binding | |
| protein P88) (P90) (IP90) | |
| kelch-like 4, isoform 1 | gi\|17017982\|ref\|NP_061990.2\|[17017982] |
| kelch-like 4, isoform 2 | gi\|17017980\|ref\|NP_476503.1\|[17017980] |
| CDC42-binding protein kinase beta; | gi\|16357474\|ref\|NP_006026.2\|[16357474] |
| CDC42-binding protein kinase beta | |
| (DMPK-like); MRCK beta; DMPK- | |
| like | |
| heat shock factor binding protein 1 | gi\|4557647\|ref\|NP_001528.1\|[4557647] |
| KIAA1052 protein | gi\|7662464\|ref\|NP_055771.1\|[7662464] |
| heat shock 105 kD; heat shock | gi\|5729879\|ref\|NP_006635.1\|[5729879] |
| 105 kD alpha; heat shock 105 kD beta | |
| nucleophosmin/nucleoplasmin 3 | gi\|16118247\|gb\|AAL12172.1\|[16118247] |
| crystallin, alpha A; human alphaA- | gi\|4503055\|ref\|NP_000385.1\|[4503055] |
| crystallin; crystallin, alpha-1 | |
| crystallin, alpha B; crystallin, alpha- | gi\|4503057\|ref\|NP_001876.1\|[4503057] |
| 2; Rosenthal fiber component; heat- | |
| shock | |
| 20 kD like-protein | |
| calnexin - human | gi\|2134858\|pir\|\|I53260[2134858] |
| calnexin | gi\|10716563\|ref\|NP_001737.1\|[10716563] |
| heat shock protein 75 | gi\|2865466\|gb\|AAC02679.1\|[2865466] |
| calnexin | gi\|179832\|gb\|AAA21749.1\|[179832] |

Preferably, such a chaperone is a protein that is highly expressed and present in the target cell population being assayed. Chaperones that are highly expressed permit the screening of candidate cyclic peptides without disrupting or interfering with the other biological activities of the particular chaperone, as the cyclic peptide will bind only a small fraction of the chaperone molecules present in the cell.

The chaperone may be ubiquitous or non-ubiquitous. Chaperones that are non-ubiquitous may provide advantages in identifying therapeutic cyclic peptides having activities target to specific cell types. Upon administration of the cyclic peptide to a human or animal, active complexes will form only in those cell types expressing the specific chaperone bound by the cyclic peptide. Cells that do hot express the chaperone will be unaffected by the presence of the cyclic peptide.

One class of highly expressed proteins that are ideal for use as chaperones in the present methods is the immunophinns. As discussed in the Background section, immunophilins, which include FK binding proteins and cyclophilins, are known to bind immunosuppressant cyclic peptides or macrocycles, such as FK506, Rapamycin (“Rap”) or cyclosporin A (“CsA”). Such immunophilin-drug binary complexes inhibit or interfere with signal transduction cascades by acting on specific effector molecules. For example, both CsA-CypA and FK506-FKBP complexes inhibit transcription of lymphokine genes and consequent activation of T-cells. The target of these complexes has been identified as the Ca²⁺/calmodulin dependent serine/threonine phosphatase calcincurin (Liu et al., 1991, Cell 66:807; Liu et al., 1992, Biochemistry 31:3896; Flanagan et al., 1992, Nature 352:803; McCaffrey et al., 1993, J. Biol. Chcm. 268:3747; McCafrey et al., 1993, Science 262:750). Likewise, Rap-FKBP complexes interfere with mitogen-induced T-cell activation, B-cell proliferation and proliferation induced by several cytokines, including IL-2, IL-3, IL-4 and IL-6 (Seghgal et al., 1994, Med. Res. Rev. 14:1-22); interfere with a calcium independent signaling cascade in T-cells and mast cells (Schreiber et al., 1992, Tetrahedron 48:2545-2558); inhibit cytokine-induced activation of p70⁵⁶ linase (Calvo et al., 1992, Proc. Natl. Acad. Sci. USA 89:7571-7575; Chung et al., 1992, Cell 69:1227-1236; Kuo et al., 1992, Nature 358:70-73; Price et al., 1992, Science 257:973-977); and inhibit activation of cdk2/cyclin E complex (Flanagan et al., 1993, J. Cell Biochem. 17A:292). The specificity of the immunophilin-drug complexes is presumed to be due to the bound drug. In particular, as exemplified by the above discussion, while the FK binding protein FKBP12 (described in more detail, below) binds both FK506 and Rap to form biologically active complexes, the respective complexes have different targets. As discussed in the Background Section, structure similarities between the regions of IP3R, FK506 and Rap that bind FKBP12 tend to confirm this theory.

Moreover, the immunophilins are highly expressed proteins. The activities of FK506, Rap and CsA are known to require only a small fraction of their respective available immunophilins. For example, in the case of FK506 and Rap, it is estimated that these compounds effect their biological activities by binding only 1/300th of the expressed FK binding protein. Thus, the immunophilins make ideal chaperones to identify cyclic peptides intended for therapeutic applications. When administered to humans or animals, such identified cyclic peptides need only bind a small fraction of the available immunophilin, leaving the remainder of the immunophilin to carry out its other biological functions.

Numerous immunophilins that can be used as chaperones in accordance with the principles of the invention are known in the art. The family of currently known FK560 binding proteins (KBPs), which is characterized by FKBP12 (see, e.g., Harding et al., 1989, Nature 341:758-760; Siekierka et al., 1989, Nature 341:755-757; European Patent Application No. 0 379 342, the latter two of which set forth the nucleotide and deduced amino acid sequence of cDNA encoding FKBP12) include FKBPP13 (see, e.g., U.S. Pat. No. 5,498,597), FKBP12.6, FKBP25, FKBPr38, FKBP51 and FKBP52. These various FKBPs, as well as their various biological activities and targets, are reviewed in Armistead & Harding, 1993, Ann. Rep. Med. Chenm. 28:207-215. Any of these FKBPs, or later-discovered FKBPs, may be used as a chaperone in connection with the methods of the invention.

The family of currently-known cyclophilins include CypA, CypB, CypC and CypD. The cyclophilins are reviewed in, e.g., Bukrinsky, 2002, Trends lmmunol. 23(7):323-325 and Andreeva et al., 1999, Int. J. Exp. Pathol. 80(6):305-315. Any of these cyclophilins, or later-discovered cyclophilins, may be used as a chaperone in connection with the methods of the invention.

Referring to FIG. 1, at a minimum, candidate cyclic peptides comprise two regions: a chaperone binding region 112 and a putative target binding region 114. The sequence of the chaperone binding region will depend upon the specific chaperone, and will be apparent to those of skill in the art. For example, the sequence of the chaperone binding region may be designed to correspond to a peptide or region of a polypeptide known to bind the specific chaperone selected. Alternatively, the sequence may be designed to correspond to conserved or consensus sequences known (or suspected) to bind the specific chaperone selected. As another example, if the three dimensional structure of the chaperone is available, a chaperone binding region can be designed in silico based upon available structural information.

Alternatively, the chaperone binding sequence may be determined from binding experiments carried out with the chaperone. In one embodiment, the chaperone is contacted with a library of peptides to identify those peptides of the library that bind the chaperone. The sequence of the chaperone binding region of the candidate cyclic peptides can correspond to the sequence of an identified peptide. If there are known ligands for the chaperone, the binding experiments may be carried out in a competitive fashion to identify those peptides that competitively bind the chaperone. For example, peptides that bind an FKBP in approximately the same region as FK506 can be identified in competitive binding experiments with an FKBP-FK506 complex. Peptides that bind a cyclophilin at approximately the same region as CsA may be identified in competitive binding experiments with a CypA-CsA complex. Myriad techniques and methods for screening libraries of peptides for the ability to bind a specific target protein of interest and for carrying out competitive binding assays that can be used to identify chaperone binding sequences suitable for use in the method of the invention are known in the art. Any of these techniques may be employed to identify and/or design chaperone binding sequences.

One art-known approach uses recombinant bacteriophage to produce large libraries of peptides which can then be screened in a variety of formats for binding to a specific chaperone of interest. Methods for constructing and screening such “phage display” libraries are described, for example, in Scott & Smith, 1990, Science 249:386-390; Cwirla et al., 1990, Proc. Natl. Acad. Sci. 87:6378-6382; 1990); Devlin et al., 1990, Science 249:404-406 (1990); U.S. Pat. No. 5,427,908; U.S. Pat. No. 5,432,018; U.S. Pat. No. 5,580,717 and U.S. Pat. No. 5,723,286, the disclosures of which are incorporated herein by reference. Other non-limiting examples of recombinant library methodologies that may be used to identify peptides that bind a chaperone of interest are described in U.S. Pat. No. 6,156,571; U.S. Pat. No. 6,107,059 and U.S. Pat. No. 5,733,731, the disclosures of which are incorporated herein by reference.

A second art-known approach uses chemical methods to synthesize libraries of compounds, such as small organic compounds, peptides and/or peptide analogs, attached to beads or wafers that can then be conveniently screened for binding with a chaperone of interest. The libraries may be encoded or non-encoded. Methods of synthesizing such immobilized libraries, as well as methods of screening the libraries are described, for example, in Houghten, 1985, Proc. Natl. Acad. Sci. USA 82:5131-5735; Geysenetal., 1986, Molecular Immunology 23:709-715; Geysen et al., 1987, J. Immunologic Method 102:259-274; Frank & Dbring, 1988, Tetrahedron 44:6031-6040; Fodor et al., 1991, Science 251:767-773; Furka et al., 1988, 4th International Congress of Biochemistry, Volume 5, Abstract FR:013; Furka, 1991, Int. J. Peptide Protein Res. 37:487-493; Frank, 1992, Tetrahedron 48:9217-9232; Needels et al., 1993, Proc. Natl. Acad. Sci. USA 90:10700-10704; DeWitt et al., 1993, Proc. Natl. Acad. Sci. USA 90:6909-6913; Frank et al., 1993, Biorg. Med. Chem. Lett. 3:425-430; Ohlmeyer et al., 1993, Proc. Natl. Acad. Sci. USA 90:10922-10926; WO 92/00252; WO 9428028; U.S. Pat. No. 6,329,143; U.S. Pat. No. 6291,183; U.S. Pat. No. 5,885,837; U.S. Pat. No. 5,424,186; U.S. Pat. No. 5,384,261; U.S. Pat. No. 6,165,717; U.S. Pat. No. 6,143,497; U.S. Pat. No. 6,140,493; U.S. Pat. No. 5,789,162; U.S. Pat. No. 5,770,358; U.S. Pat. No. 5,708,153; U.S. Pat. No. 5,639,603; U.S. Pat. No. 5,541,061; U.S. Pat. No. 5,525,735; U.S. Pat. No. 5,525,734; U.S. Pat. No. 6,261,776; U.S. Pat. No. 6,239,273; U.S. Pat. No. 5,846,839; U.S. Pat. No. 5,770,455; U.S. Pat No. 5,770,157; U.S. Pat. No. 5,609,826; U.S. Pat. No. 6,001,579; U.S. Pat. No. 5,968,736; U.S. Pat. No. 5,962,337; U.S. Pat. No. 5,789,172; U.S. Pat. No. 5,721,099; U.S. Pat. No. 5,565,324; U.S. Pat. No. 5,010,175; and U.S. Pat. No. 4,631,211, the disclosures of which are incorporated herein by reference. For reviews of some of these techniques, see Ellman et al., 1996, Account, Chem. Res. 29:132-143; Gallop et al., 1994, J. Med. Chem. 37:1233-1251; Gordon et al., 1994, J. Med. Chem. 37:1385-1401. Additional review articles, references, patents and books describing myriad techniques for synthesizing and screening libraries of peptides for the ability to bind a chaperone of interest can be found at Lebl & Leblova: Dynamic Database of References in Molecular Diversity, Internet http://www.5z.com (see especially the diversity information pages at htto://www.5z.com/divifo).

In an alternative embodiment, sequences suitable for use in the chaperone binding region can be identified using the power of the immune system. For example, antibodies specific for the chaperone can be generated using standard techniques (described in more detail, below) and the sequence(s) of the antigenic regions or portions determined. These sequences can then be used to define the sequence of the chaperone binding region of candidate cyclic peptides. Skilled artisans will recognize that myriad techniques for generating antibodies and/or fragments of antibodies that specifically bind molecules are well-known in the art. Myriad library techniques for identifying antibodies, antibody fragments and/or single-chain variants thereof are also known in the art. Any of these methods may be used to identify sequences useful for the chaperone binding region of the candidate cyclic peptides. Some of these various immunological methods are described in more detail, below.

In a preferred embodiment, the chaperone binding region includes a sequence capable of binding an immunophilin. A specific sequence capable of binding the inununophllin CypA that may be used in connection with the invention is Ala-Gly-Pro-Ile. A specific sequence capable of binding the immunophilin FKBP12 that may be used in connection with the invention is Leu-Pro.

Referring to FIG. 1, the candidate cyclic peptides also include a putatitive target binding region. The putative target binding region constitutes a “variable” region of the candidate cyclic peptide. The sequence of this region can be completely random. For example, the sequence of the putative target binding region may include a random peptide sequence composed of 2, 4, 5, 6, 7, 8, 9, 10, 11, 12, 13, 14, 15, 16, 17, 18, 19, 20, or even more amino acid residues. Alternatively, the sequence can include one or more variable or random regions flanked by one or more regions of defined sequence(s). As a specific example, the sequence of the putative binding region may include a region of defined sequence disposed between two variable regions, or vice versa. The region(s) of defined sequence(s) can be selected to reduce the complexity of the library or for other reasons, such as, for example, to perform specified functions. For example, the defined sequences can be selected so as to restrict conformationally any variable or random sequences fused thereto. A variety of different defined sequences that can be included in the target binding region of the candidate cyclic peptides are described in U.S. Pat. No. 6,153,380 (see especially Cols. 4 12), the disclosure of which is incorporated herein by reference. These sequences can be used alone or in various combinations. The regions of defined sequence(s) can be fused directly to the random or variable regions, or they may be spaced away from such regions via one or more of the same or different linkers (described in more detail, below).

In a preferred embodiment, the putative binding region is a random sequence ranging from 2 to 20 amino acids in length, more preferably from 2 to 8 amino acids in length.

As illustrated in FIG. 1, the chaperone binding and putative target binding regions are fused together, optionally by way of linkers 116. When linkers are used, they may be the same or different, and may vary widely in properties. For example, the linkers may be flexible or rigid, hydrophobic or hydrophilic or long or short, depending upon a particular application. Preferably, any optional linkers used will be flexible. Suitable flexible linkers include polyglycine, glycine-serine polymers, including, for example, (GS)ₙ, (GSGGS)ₙ and (GGGS)ₙ where n is an integer of at least one, typically from 1 to 2; glycine-alanine polymers, alanine-serine polymers, and other flexible linkers such as the tether for the shaker potassium channel, and a large variety of other flexible linkers, as will be appreciated by those in the art.

The candidate cyclic peptides may be synthesized chemically and administered to the cells by contacting the cells with the candidate cyclic peptide. Myriad techniques for synthesizing cyclic peptides are well-known (see, e.g., Tam et al., 2000, Biopolymers 52:311-332; Camamero et al, 1998, Angew. Chem. Intl. Ed. 37: 347-349; Tam et al., 1998, Prot. Sci. 7:1583-1592; Jackson et al., 1995, J. Am. Chem. Soc. 117:819-820; Dong et al., 1995, J. Am. Chem. Soc. 117:2726-2731; Ishida et al., 1995, J. Org. Chem. 60:5374-5375; WO 95/33765, published Jun. 6, 1995; Xue and DeGrado, 1994, J. Org. Chem. 60(4):946-952; Jacquier et al., 1991, In: Peptides 1990 221-222, Giralt and Andreu, Eds., ESCOM Leiden, The Netherlands; Schmidt and Neubert, 1991, In: Peptides 1990 214-215, Giralt and Andreu, Eds., ESCOM Leiden, The Netherlands; Toniolo, 1990, Int. J. Peptide Protein Res. 35:287-300; Ulysse etal., 1995, J. Am. Chem. Soc. 117:8466-8467; Durr et al., 1991, Peptides 1990 216-218, Giralt and Andreu, Eds., ESCOM Leiden, The Netherlands; Lender et al., 1993, Int. J. Peptide Protein Res. 42:509-517; Boger and Yohannes, 1990, J. Org. Chem. 55:6000-6017; Brady et al., 1979, J. Org. Chem. 4(18):3101-3105; Spatola et al., 1986, J. Am. Chem. Soc. 108:825-831; Seidel et al., 1991, In: Peptides 1990 236-237, Giralt and Andreu, Eds., ESCOM Leiden, The Netherlands; Tanizawa et al., 1986, Chem. Phar, Bull. 34(10):4001-4011; Goldenburg & Creighton, 1983, J. Mol. Biol. 165:407-413). The cyclic peptides can be synthesized on a compound-by-compound basis, or, altematively, the cyclic peptides may be synthesized as libraries by routine adaptation any of the myriad known methodologies for synthesizing libraries of compounds. Addition methods that may routinely adapted to the synthesis of cyclic peptide libraries are described in the art (see, e.g., Bunin, 1998, The Combinatorial Index, Academic Press, San Diego, Calif., and the various references cited therein).

Preferably, however, the candidate cyclic peptides are administered to the cells by way of candidate polynucleotides capable of expressing the cyclic peptides inside the cell. A variety of polynucleotides capable of expressing cyclic peptides in cells that utilize the trans splicing ability of inteins have been developed. Such polynucleotides can be used to administer candidate cyclic peptides to cells in accordance with the method of the invention

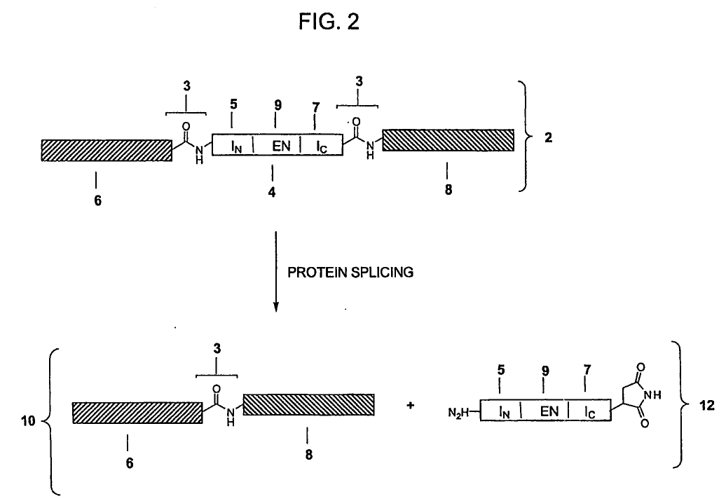

Inteins are protein splicing elements that mediate their excision from precursor proteins and the joining of the flanking proteins (N-extein and C-extein) to yield two mature proteins: the intein and the ligated protein (Perler et al., 1994, Nucl. Acids Res. 22:1125-1127). The bond formed between the ligated exteins is a native peptide bond (Perler et al., 1997, Curr. Opin. Chem. Biol. 1:292-299). The self-catalytic splicing reaction requires four nucleophilic displacements mediated by three conserved splice junction residues: (i) a Ser, Crys or Ala at the intein N-terminus; (ii) an Asn or Asp at the intein C-terminus; and (iii) a Ser, Thr or Cys at the beginning of the C-extein (Xu et al., 1993, Cell 75:1371-1377; Xu et al., 1994, EMBO J. 13:5517-5522; Shao et al. 1995, Biochemistry 34:10844-10850; Chong et al., 1996, J. Biol. Chem. 271:22159-22168; Xu & Perler, 1996, EMBO J. 15:5146-5153). An example of an intein-mediated protein splicing reaction is illustrated in FIG. 2. Referring to FIG. 2, precursor protein 2, which comprises an intein 4 flanked by an N-terminal extein 6 and a C-terminal extein 8, undergoes a protein splicing reaction, which is mediated or catalyzed by intein 4, to yield the excised intein 12 and fusion protein 10. As illustrated, intein 4 comprises an N-terminal protein splicing domain 5 (IN) and a C-terminal protein splicing domain 7 (Ic) flanking an endonuclease domain 9 (EN). Fusion protein 10 comprises N-terminal extein 6 fused to C-terminal extein 8 via a native peptide bond 3.

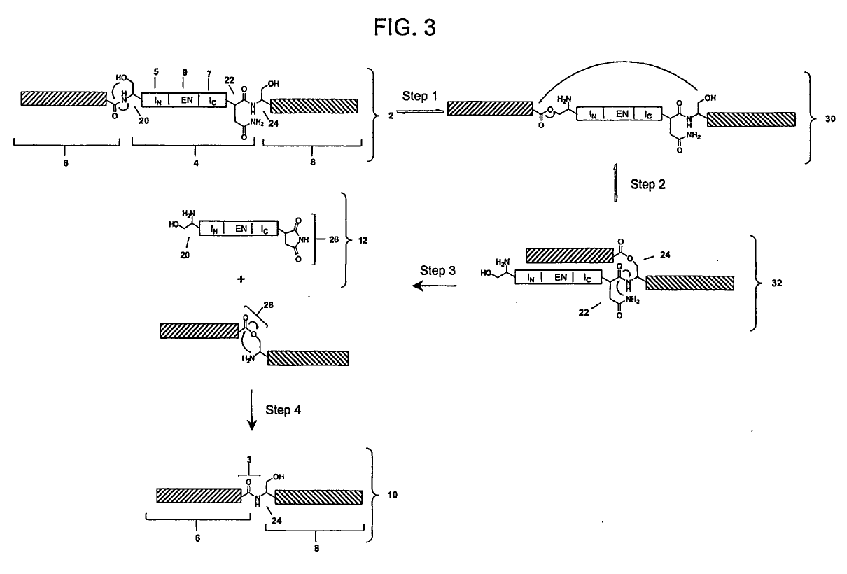

The mechanism of action of the intein-mediated protein splicing reaction is illustrated in FIG. 3. Referring to FIG. 3, which illustrates the precursor protein 2 of FIG. 2 showing a required Ser 20 at the intein N-terminus, the required Asn 22 at the intein C-terminus, and a required Ser 24 at the N-terminus (beginning) of the C-terminal extein 8, the splicing reaction proceeds via four steps: (1) Step 1 is an N—O acyl shift that occurs between the C-terminal amino acid of N-terminal extein 6 and the N-terminal Ser 20 of intein 4, resulting in the formation of a reactive ester 30; (2) in Step 2, the ester 30 is the focus of a transesterfication reaction that results in a branched intermediate 32 in which N-terminal extein 6 is attached via an ester to the N-terminal Ser 24 of C-terminal extein 8; (iii) in Step 3, branched intermediate 32 is resolved by the cyclization of the intein C-terminal Asn residue 22 to form a succinimide group 26, the excised intein 12 is released from precursor 2 and N-termial extein 6 and C-terminal extein 8 are joined by an ester bond 28; (iv) in Step 4, a spontaneous O—N acyl shift generates a native peptide bond 3 between exteins 6 and 8 at the condensation point to yield fusion protein 10.

Split inteins, whether naturally occurring or artificially created by splitting a contiguous intein into two distinct “halves,” are capable of catalyzing a trans ligation reaction that yields an extein product cyclized in a head-to-tail fashion (see, e.g., Southworth et al., 1998, EMBO J. 17:918-926; Xu et al., 1999, Proc. Natl. Acad. Sci. USA 95:6705-6710; Evans et al., 1999, Biochemistry 274:18359-18363; Scott et al., 1999, Proc. Natl. Acad. Sci. USA 96:13638-13643). The splitting of an intein to catalyze a .Irans ligaLou reaction generally requires segregating the I_{N} and I_{C} protein splicing domains and reversing their translational order such that the splicing and ligation reaction fuses the former N— and C-termini. This process is well-understood and is described, for example, in WO 01/66565 (see, e.g., FIGS. 1A, 1B, 2A and 2B and the text associated therewith).

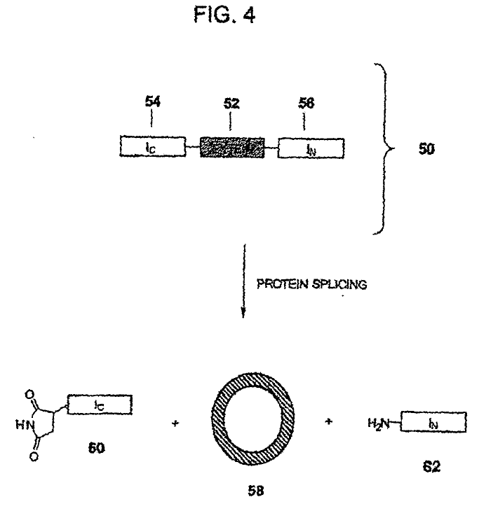

An example of such a split intein construct and the cyclization reaction catalyzed thereby is illustrated in FIG. 4. Referring to FIG. 4, precursor protein 50 comprises an extein 52 interposed between two intein protein splicing domains 54, 56. The upstream intein domain 54 corresponds to the C-terminal (I_{C}) domain 7 of FIG. 2. The downstream intein domain 56 corresponds to the N-terminal (I_{N}) domain 5 of FIG. 2.

The intein illustrated in FIG. 2 is one of many inteins that is bifimctional in that it mediates both protein splicing and DNA cleavage. This latter activity is mediated by the endonuclease domain (EN) 9 that interrupts the N— and C-terminal intein protein splicing domains 5, 7 illustrated in FIG. 2. Because the endonuclease activity is not required for protein splicing, split intein constructs capable of expressing cyclic peptides that are designed from such bifunctional inteins need not include the endonuclease domain (see, e.g., Wood et al., 1999, Nature Biotechnology 17:889-892). Thus, the precursor protein 50 illustrated in FIG. 4 does not include an endonuclease domain.

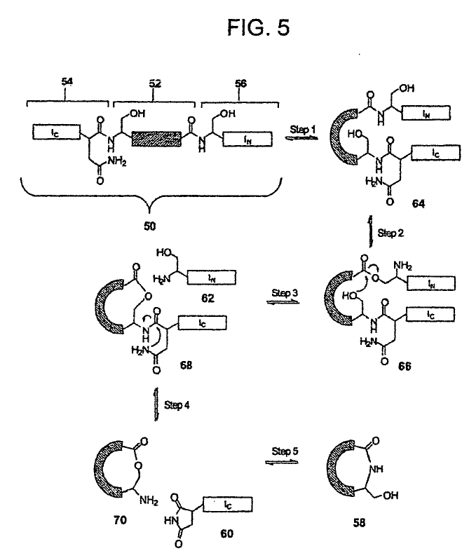

Referring again to FIG. 4, when such a precursor protein 50 is expressed in an appropriate host system, I_{C} 54 and I_{N} 56 physically come together to form an active intein (illustrated in FIG. 5) that catalyzes a protein splicing reaction that yields an extein cyclized in a head-to-tail fashion 58 and released C— and N-terminal intein domains 60 and 62. Like the splicing reaction catalyzed by the contiguous intein illustrated in FIGS. 2 and 3, the splicing reaction catalyzed by the split intein construct 50 of FIG. 4 also requires four nucleophilic displacements mediated by three conserved splice junction residues: (i) a Ser, Cys or Ala at the N-terminus of I_{N} 56; (ii) an Asn or Asp at the C-terminus of I_{C} 54; and (iii) a Ser, Cys or Thr at the N-terminus beginning) of extein 52

The mechanism of the trans splicing reaction is illustrated in FIG. 5. In FIG. 5, the C-terminal Asn residue of I_{C} 54, the N-terminal Ser residue of extein 52 and the N-terminal Ser residue of I_{N} 56 are illustrated. In Step 1, I_{C} 54 and I_{N} 56 come together physically to yield an active intein 64 having a conformation that forces extein 52 into a loop configuration. In Step 2, the N-terminal Ser of I_{N} 56 undergoes an O—C acyl migration to yield a reactive ester 66. In Step 3, the oxygen of the N-terminal Ser residue of extein 52 reacts with the ester to yield a lariat product 68 and released I_{N} 62. The active intein then resolves the lariat 68 via the formation of a succinimide that liberates a cyclized lactone form of the extein 70 and I_{C} 60. The lactone 70 then spontaneously rearranges to form the thermodynamically favored head-to-tail lactone form of the cyclic peptide 58.

Candidate polynucleotides useful in the methods of the invention exploit this trans splicing ability of split inteins to express candidate cyclic peptides in cells. Thus, the candidate polynucleotides generally comprise a first segment encoding a C-terminal intein protein splicing domain (I_{C}), a second segment encoding a linear version of a candidate cyclic peptide and a third segment encoding an N-terminal intein protein splicing domain (I_{N}). The three segments are arranged such that when expressed, the polynucleotide yields the precursor protein 50 (FIG. 4) in which extein 52 corresponds to the linear version of a candidate cyclic peptide. Precursor protein 50 then undergoes spontaneous splicing to yield the candidate cyclic peptide (element 58 of FIG. 4).

Nucleotide sequences that encode I_{C} and I_{N} may be derived from naturally-occunring split inteins (ie., inteins that in nature are produced as two distinct polypeptide chains) or from contiguous inteins that have been artificially split and rearranged using known techniques. One example of a naturally-occurring split intein that may be used in connection with the polypeptides of the invention is Ssp DnaE (Wu et al., 1998, Proc. Natl. Acad. Sci. USA 95:9226). Another specific example is Ssp DnaB (Wu et al., 1998, Biochem Biophys Acta 1387:422-432; Evans et al., 1999, J. Biol. Chem. 274:18359-63). A fairly comprehensive list of contiguous inteins that may be artificially split and rearranged to express candidate cyclic peptides according to the invention is published by New England Biolabs at http://www.neb.com/inteins/int_req.html. Guidance for splitting and rearranging such inteins for use in the candidate polynucleotides of the invention may be found, for example, in WO 01/66565; Evans et al., 1999, J. Biol. Chem. 274:18359; Mills et al., 1998, Proc. Natl. Acad. Sci. USA 95:3543. Specific examples of contiguous inteins that may be split and rearranged in accordance with the invention include Psp-Pol-1 (Southworth et al., 1998, EMBO J. 17:918), Mycobacterium tuberculosis RccA intein (Lew et al., 1998, J. Biol. Chem. 273:15887; Shingledecker et al., 1998, Gene 207:187; Mills et al., 1998, Proc. Natl. Acad. Sci. USA 95:3543), Ssp/DnaB/MxeGyrA (Evans et al., 1999, J. Biol. Chem. 274:18359) and Pfu (Otoma et al., 1999, Biochemistry 38:16040; Yamazaki et al., 1998, J. Am. Chem. Soc. 120:5591). Additional specific examples are found in WO 01/66565. All of these various references and the inteins described therein are incorporated herein by reference.

According to the methods of the invention, the candidate polynucleotides are introduced into cells to screen for and identify expressed candidate cyclic peptides that alter a phenotype of the cells. By “introduced into” or grammatical equivalents thereof is meant that the candidate polynucleotides enter the cells in a manner suitable for subsequent expression of the candidate cyclic peptides. The method of introduction is largely dictated by the targeted cell type, discussed in more detail below. Exemplary methods include CaPO₄ precipitation, liposome fusion, lipofectin®, electroporation, viral infection, etc. The candidate polynucleotides may stably integrate into the genome of the host cell (for example, with retroviral introduction, outlined below), or may exist either transiently or stably in the cytoplasm (i.e., through the use of traditional plasmids, utilizing standard regulatory sequences, selection markers, etc.). As many pharmaceutically important screens require human or model (e.g., murine, simian, etc.) mammalian target cells the use of retroviral vectors capable of transfecting such targets are preferred.

In one embodiment, the candidate polynucleotides are part of a retroviral particle which infects the cells. Generally, infection of the cells is straightforward with the application of the infection-enhancing reagent polybrene, which is a polycation that facilitates viral binding to the target cell. Infection can be optimized such that each cell generally expresses a single construct, using the ratio of virus particles to number of cells. Infection follows a Poisson distribution.

In another embodiment, the candidate polynucleotides are introduced into the cells using the well-known retroviral vectors and infection techniques pioneered by Richard Mulligan and David Baltimore with Psi-2 lines and analogous retroviral packaging systems based upon NIH 3T3 cells (see Mann et al., 1993, Cell 33:153-159, the disclosure of which is incorporated herein by reference). Such helper-defective packaging cell lines are capable of producing all of the necessary trans proteins (gag, pol and env) requ ired for packaging, processing, reverse transcribing and integrating genomes. Those RNA molecules that have in cis the ψ packaging signal are packaged into maturing retrovirions. Virtually any of the art-known retroviral vectors and/or transfection systems may be used. Specific non-limiting examples of suitable transfection systems include those described in; WO 01/66565, WO 00/36093; WO 97/27213; WO 97/27212; Choate et al., 1996, Human Gene Therapy 7:2247-2253; Kinsella et al., 1996, Human Gene Therapy 7:1405-1413; Hoffmann et al., 1996, Proc. Natl. Acac. Sci. USA 93:5185-5190; Kitamura et al., 1995, Proc. Natl. Acac. Sci. USA 92:9146-9150; WO 94/19478; Pear et al., 1993, Proc. Natl. Acac. Sci. USA 90:8392-8396; Mann et al., 1993, Cell 33:153-159 and the references cited in all of the above, the disclosures of which are incorporated herein by reference.

In one embodiment of the invention, a library of candidate polynucleotides is generated in a retrovirus DNA construct backbone, as is generally described in U.S. Pat. No. 6,153,380 (see, e.g., Cols. 16-17) and WO 01/66565, the disclosures of which are incorporated herein by reference. Any number of suitable retroviral vectors may be used. Generally, the retroviral vectors may include: selectable marker genes under the control of internal ribosome entry sites (IRES), which allows for bioistronic operons and thus greatly facilitates the selection of cells expressing candidate cyclic peptides at uniformly high levels; and promoters driving expression of a second gene, placed in sense or anti-sense relative to the 5′ LTR. Suitable selection genes include, but are not limited to, neomycin, blastocidin, bleomycin, puromycin, and hygromycin resistance genes, as well as self-fluorescent markers such as green or blue fluorescent protein, enzymatic markers such as lacZ, and surface proteins such as CD8, etc. Specific non-limiting examples of suitable retroviral vector systems include vectors based upon murine stem cell virus (MSCV) as described in Hawley et al., 1994, Gene Therapy 1:136; vectors based upon a modified MGF virus as described in Rivere et al., 1995, Genetics 92:6733; pBABE as described in WO 97/27213 and WO 97/27212; the vectors depicted in FIG. 11 of WO 01/34806 and the vectors described in WO 01/66565 and WO 00/36093, the disclosures of which are incorporated herein by reference.

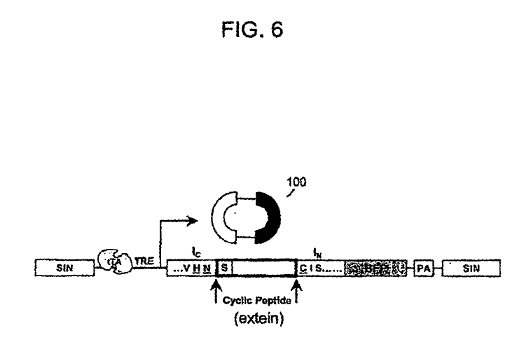

A specific example of a retroviral vector useful for creating a candidate polynucleotide library and expressed candidate cyclic peptide library is illustrated in FIG. 6. Referring to FIG. 6, the construct includes retroviral mutated non-functional LTR promoters (“SIN”) flanking an inverted intein (I_{C} and I_{N}). The illustrated amino acid of the intein are based upon the sequence of the inverted DnaB intein (Scott et al., 2001, Chem. Biol. 8:801-815). The I_{C} and I_{N} domains of the inverted intein flank the region encoding the cyclic peptide (library insert; extein). As discussed previously, inteins require nucleophilic residues after each splice junction for splicing activity at the junction (Nathys et al., 1999, Gene 231:1-13) and have an invariant Asn residue at the C-terminus of I_{C}. In FIG. 6, the invariant Ser residue is boxed, and the required nucleophilic residues Cys, Asn and His of I_{N} and I_{C} are underlined. The candidate cyclic peptide 100 expressed by this construct is illustrated above the vector. Also illustrated are an optional tetracycline-regulated transactivator (tTA) useful for regulating the expression of the cyclic peptide with tetracycline or doxycycline (discussed in more detail below) and an optional blue fluorescent protein (BFP) useful for monitoring introduction of the polynucleotides (and subsequent expression thereof) into cells.

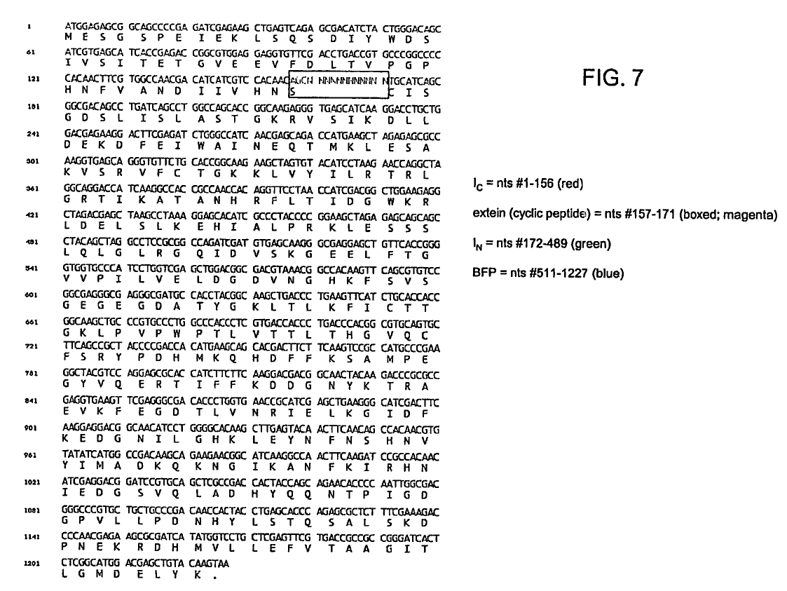

A specific example of a nucleotide sequence encoding the I_{C}-extein-I_{N} region of the construct of FIG. 6 is provided in FIG. 7. In FIG. 7, nucleotides #1-156 correspond to the I_{C} region of FIG. 6, nucleotides #157-171 correspond to the cyclic peptide (extein nucleotides #147-149 encode a fixed Ser residue) region of FIG. 6 and nucleotides #172-489 correspond to the I_{C} region of FIG. 6. Skilled artisans will recognize that in the candidate cyclic peptide (extein) region of FIG. 7, “N” represents nucleotides encoding the candidate cyclic peptides (an AGC codon encoding a Ser residue necessary for splicing is illustrated). The translated amino acids are shown below their respective codons. Stop codons are indicated with a period (“.”). Nucleotides #511-1227 encode BFP.

Another specific example of a retroviral vector that may be routinely adapted to create a candidate polynucleotide library is illustrated in U.S. Pat. No. 6,153,380, FIG. 4, the disclosure of which is incorporated herein by reference. Skilled artisans will appreciate that a polynucleotide sequence capable of expressing a candidate cyclic peptide, such as the polynucleotide sequence of FIG. 7, may be inserted into the illustrated vector at the region labeled “peptides are inserted here.”

The retroviral may include inducible and constitutive promoters. For example, there are situations wherein it may be desirable or necessary to induce cyclic peptide expression only during certain phases of the selection process. In such instances, the cyclic peptide expression is only turned on during that phase of the selection desired. A large number of both inducible and constitutive promoters that may be used in this context are well-known.

In addition, it is possible to configure a retroviral vector to allow inducible expression of retroviral inserts after integration of a single vector in target cells; importantly, the entire system is contained within the single retrovirus. Tet-inducible retroviruses have been designed incorporating the Selfinactivating (SIN) feature of 3′ LTR enhancer/promoter retroviral deletion mutant (Hoffinan et al., 1996, PNAS USA 93:5185). Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, indicating that expression is regulated uniformly within the infected cell population. A similar, related system uses a mutated Tet DNA-binding domain such that it bound DNA in the presence of Tet, and was removed in the absence of Tet. Either of these systems is suitable for use in connection with the present invention.

Delivery of the candidate polynucleotide library into a retroviral packaging system results in conversion to infectious virus. Suitable retroviral packaging system cell lines include, but are not limited to, the Bing and BOSC23 cell lines described in WO 94/19478; Soneoka et al., 1995, Nucleic Acid Res. 23(4):628; Finer et al., 1994, Blood 83:43; Phoenix packaging lines such as PhiNX-eco and PhiNX-ampho, described in U.S. Pat. No. 6,153,380 (see, e.g., Col 18, line 9 through Col. 19, line 29); 292T+gag-pol and retrovirus envelope; PA317; and cell lines outlined in Markowitz et al., 1988, Virology 167:400, Markowitz et al., 1988, J. Virol. 62:1120, Li et al., 1996, PNAS USA 93:11658, Kinsella et al., 1996, Human Gene Therapy 7:1405, WO 01/66565, WO 00/36093, in WO 97/27213 and WO 97/27212, all of which are incorporated by reference. If necessary, the virus may be concentrated as described in U.S. Pat. No. 6,153,380 (see, e.g., Col. 19, lines 35-61), the disclosure of which is incorporated herein by reference.

The candidate polynucleotides, as part of the retroviral construct, are introduced into a plurality of cells to screen for candidate cyclic peptides capable of altering a phenotype of a cell. By a “plurality of cells” is meant roughly from about 10³ cells to 10⁸ or 10⁹ cells, with from about 10⁶ to 10⁸ cells being typical. This plurality of cells comprises a cellular library, wherein generally each cell within the library contains a member of the retroviral construct library, i.e. a different candidate polynucleotide, although as will be apprcclated by those in the art, some cells within the library may not contain a retrovirus, and some may contain more than one. When methods other than retroviral infection are used to introduce the candidate polynucleotides into a plurality of cells, the distribution of candidate polynucleotides within the individual cell members of the cellular library may vary widely, as it is generally difficult to control the number of nucleic acids which enter a cell during electroporation, etc.

The types of cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. Typically, a screen will be set up such that the cells exhibit a selectable phenotype in the presence of a candidate cyclic peptide. Skilled artisans will be able to select an appropriate or suitable cell type depending upon, among other factors, the chaperone targeted by the chaperone binding region of the candidate cyclic peptides and the types of biological processes regulated by the chaperone. Cell types implicated in a wide variety of disease conditions are particularly useful, so long as a suitable screen may be designed to allow the selection of cells that exhibit an altered phenotype as a consequence of the presence of a candidate cyclic peptide within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO cells, Cos cells, etc., and the myriad other cell lines described in the ATCC cell line catalog, incorporated by reference. As specific examples, T-cells, B-cells, mast cells and other types of immune system cells are suitable for candidate cyclic peptides including chaperone binding regions that bind imrunophilins.

The cells in which the candidate polynucleotides are screened may be optionally genetically engineered to contain exogenous nucleic acids. For example, the cells may be engineered to include a reporter gene (e.g., a gene encoding a fluorescent protein) fused downstream of a specific responsive element or promoter. The selection of the particular responsive element or promoter will depend upon the particular process for which modulatory cyclic peptides are desired, and will be apparent to those of skill in the art. Altematively, or in addition, the cells may be optionally engineered to express or overexpress the chaperone. However, in preferred embodiments a cell type is selected that highly expresses the chaperone endogenously.

If necessary, the cells are treated to conditions suitable for the expression of the candidate polynucleotides (for example, when inducible promoters are used), to produce the candidate cyclic peptides. The plurality of cells is then screened, as is more fully outlined below, for a cell exhibiting an altered phenotype. The altered phenotype is due to the formation of a ternary complex between a candidate cyclic peptide, the chaperone and a target molecule, as illustrated in FIG. 1.

By “altered phenotype” or “changed physiology” or other grammatical equivalents thereof is meant that a phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density, changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytolines, receptors, or other molecules; changes in the secretion of ions, cytokines, hormones, growth factors, or other molecules; alterations in cellular membrane potentials, polarization, integrity or transport; changes in infectivity, susceptibility, latency, adhesion, and uptake of viruses and bacterial pathogens; etc. The specific phenotypic change selected for observation will depend upon a variety of factors, and will be apparent to those of skill in the art. A variety of specific biological processes and their respective phenotypes for which modulatory cyclic peptides may be identified in connection with the methods of the present invention are described in, among other references, U.S. Pat. No. 6,153,380 (see, e.g., Cols. 26-34), the disclosure of which is incorporated herein by reference.

The altered phenotype may be detected in a wide variety of ways, as is described more fully below, and will generally depend and correspond to the phenotype that is being altered. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In embodiments in which the cells have been engineered to include a reporter gene, the change in phenotype can be determined by assessing the level of expression of the reporter gene, either at the transcriptional level or the translational level.

In most cases, the altered phenotype is detected in the cell in which the candidate polypeptide was introduced (i.e., the cells are assessed for autocrine effects). However, in some instances, such as those instances where the desired phenotypic change involves a change in the ability of a cell to signal extracellularly, the altered phenotype is detected in a second cell, or even a third cell, which is responding to some molecular signal from the first cell. In this latter embodiment, the second population of cells is assessed for endocrine or paracrin effects. The first cell which received the candidate polynucleotide may or may not be assessed for an altered phenotype. Methods for observing or detecting endocrine or paracfin effects are well known.

Once a cell with an altered phenotype is detected (“positive cell”), the cell may be isolated from the plurality which do not have altered phenotypes. This may be done in any number of ways, as is known in the art, and will in some instances depend on the assay or screen. Suitable isolation techniques include, but are not limited to, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes, etc.

The candidate polynucleotide from the positive cells may be sequenced, with or without prior isolation, to determine the sequence of the putative target binding region of the expressed candidate cyclic peptide. Alternatively, the candidate cyclic peptide may be isolated and characterized using standard analytical techniques (e.g., mass spectroscopy, NMR, etc).

The identified cyclic peptide may be synthesized synthetically and introduced into the target cells to confirm its activity. Alternatively, the candidate polynucleotide may be reintroduced into the target cells to confirm the activity of the expressed cyclic peptide.

The sequence of the putative target binding region may also be used generate additional candidate cyclic peptides having altered activities, as is well-known in the art.

A significant advantage of the method of the invention is the ability to isolate from the positive cells the target molecule(s) with which the CCP-C complex interacts. Since the chaperone targeted by the chaperone binding region of the candidate cyclic peptides is know, the CCP-C complexes may be isolated from lysates of positive cells using well-known affinity techniques. As a specific example, the CCP-C complexes may be isolated from positive cell lysates using affinity chromatography with an antibody that specifically binds the chaperone.

For the production of antibodies, various host animals, including but not limited to rabbits, mice, rats, etc., may be immunized by injection with the chaperone of interest. The chaperone may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a specific chaperone may be prepared using any technique which provides for the production of antibody molecules by continuous cell lines in culture. These include, but are not limited to, the hybridoma technique originally described by Kohler & Milstein, 1975, Nature 256:495-497 and/or Kaprowski, U.S. Pat. No. 4,376,110; the human B-cell hybridoma technique described by Kosbor et al., 1983, Immunology Today 4:72 and/or Cote et al., 1983, Proc. Natl. Acad. Sci. USA 80:2026-2030; and the EBV-hybridoma technique described by Cole et al., 1985, Monoclonal Antibodies and Cancer Therapy, Alan R. Liss, Inc., pp. 77-96. In addition, techniques developed for the production of “chimeric antibodies” (Morrison et al.., 1984, Proc. Natl. Acad. Sci. USA 81:6851-6855; Neuberger et al., 1984, Nature 312:604-608; Takeda et al., 1985, Nature 314:452-454; Boss, U.S. Pat. No. 4,816,397; Cabilly, U.S. Pat. No. 4,816,567) by splicing the genes from a mouse antibody molecule of appropriate antigen specificity together with genes from a human antibody molecule of appropriate biological activity can be used. Or “humanized” antibodies can be prepared (see, e.g., U.S. Pat. No. 5,585,089). Alternatively, techniques described for the production of single chain antibodies (see, e.g., U.S. Pat. No. 4,946,778) can be adapted to produce chaperone-specific single chain antibodies.

Antibody fragments which contain deletions of specific binding sites may be generated by known techniques. For example, such fragments include but are not limited to F(ab′)2 fragments, which can be produced by pepsin digestion of the antibody molecule and Fab fragments, which can be generated by reducing the disulfide bridges of the F(ab′)2 fragments. Alternatively, Fab expression libraries may be constructed (Huse et al., 1989, Science 246:1275-1281) to allow rapid and easy identification of monoclonal Fab fragments with the desired specificity for the chaperone.

As discussed previously, these various methods can also be used to define sequences useful for the chaperone binding region of the candidate cyclic peptides.

The antibody or antibody fragment specific for the chaperone can be attached, for example, to agarose, and the antibody-agarose complex is used in immunochromatography to isolate CCP-C complexes of the invention. See, Scopes, 1984, Protein Purification: Principles and Practice, Springer-Verlag New York, Inc., NY; Livingstone, 1974, Methods In Enzymology: Immunoaffinity Chromatography of Proteins 34:723-731. The isolation is carried out under conditions conducive to binding between the CCP-C complex and any bound target molecule. Such conditions are typically physiological conditions, but may vary depending upon the cell type used to screen the candidate cyclic peptides. Any target molecule bound to the immobilized CCP-P complexes may be dissociated there from using standard techniques and characterized, again using standard techniques.

While the invention has been described by reference to various specific and/or preferred embodiments, skilled artisans will recognize that numerous modifications may be made thereto without departing from the spirit and the scope of the appended claims. As a specific example, while the methods have been exemplified with reference to cyclic peptide candidate compounds, skilled artisans will recognize that virtually any type of molecule can be screened as a candidate compound according to the methods of the invention. Thus, the compounds screened can range from small organic molecules to large polymers and biopolymers, and can include, by way of example and not limitation, small organic compounds, saccharides, carbohydrates, polysaccharides, lectins, peptides and analogs thereof, polypeptides, proteins, antibodies, oligonucleotides, polynucleotides, nucleic acids, etc. The only requirement is that the candidate compound include two distinct regions or portions: one region or portion that binds a specific chaperone, and another region or portion that can vary in structure (putative target binding region).

As another specific example, while the invention has been described in the context of intracellular processes and chaperones, skilled artisans will recognize that the principles of the invention can also be applied to the identification of cyclic peptides that interact with and modulate extracellularly processes mediated by membrane-bound receptors and/or extracellular processes. Thus, serum proteins and other molecules can also be used as chaperones according to the principles of the invention. Suitable serum molecules useful as chaperones in the methods of the invention include, but are not limited to, plasma proteins such as serum albumin, lipoproteins, immunoglobulins, fibrinogen, transferrin and alpha-i acid glycoprotein, as well as non-protein molecules such as heparin.

All references cited throughout the disclosure are incorporated herein by reference in their entireties for all purposes.

1. A method of identifying a cyclic peptide capable of altering a phenotype of a cell, comprising the step of:
administering to the cell a cyclic peptide comprising a chaperone binding region of known sequence and a target binding region of wholly or partially unknown sequence; and
assessing whether a phenotype of the cell has been altered.

2. The method of claim 1 in which the cyclic peptide is composed wholly of gene-encoded amino acids.

3. The method of claim 1 in which the chaperone binding region binds an imunophilin.

4. The method of claim 3 in which the immunophilin is cyclophilin or n FK-binding protein.

5. The method of claim 4 in which the cyclophilin is selected from the group consisting of cypA, cypB, and cycD.

6. The method of claim 4 in which the fk-binding protein is selected fromt eh group consisting of FKBP12, FKBP13, FKBP25, and FKBP59.

7. The method of claim 3 in which the immunophilin is endogenous to the cell.

8. The method of claim 3 in which the immunophilin is exogenous to the cell.

9. The method of claim 1 in which the chaperone binding region has an amino acid sequence selected from the group consisting of Ala-Gly-Pro-Ile and Leu-Pro.

10. The method of claim 1 further including the step of determining the sequence of the target binding region of the cyclic peptide.

11. The method of claim 1 in which the target binding region of the cyclic peptide is composed of from 4 to 10 amino acid residues.

12. The method of claim 1 in which the chaperone binding region and the target binding region of the cyclic peptide are contiguous.

13. The method of claim 1 in which the chaperone binding region and the target binding region of the cyclic peptide are spaced apart from one another via linkers, which may be the same or different.

14. A method of identifying a cyclic peptide capable of altering a phenotype of a cell, comprising the steps of:
administering to a plurality of cells a plurality of cyclic peptides, each of which comprises a chaperone binding region and a target binding region;
identifying those cells exhibiting an altered phenotype (positive cells); and
determining the sequence of at least the target binding region of the cyclic peptides of positive cells.

15-21. (canceled)

22. A method of isolating a target capable of altering a phenotype of a cell, comprising the steps of:
administering to a cell expressing a chaperone a cyclic peptide comprising a region capable of binding the chaperone and a target binding region of wholly or partially unknown sequence;
if the cell exhibits an altered phenotype, contacting a lysate of the cell with an affinity reagent capable of specifically binding a the chaperone; and
dissociating and isolating any target bound to the chaperone.

23-26. (canceled)

27. A method of identifying a target capable of altering a phenotype of a cell, administering to each of a plurality of cells a cyclic peptide comprising a chaperone binding region and a unique target binding region;
isolating a cell exhibiting an altered phenotype;
contacted said isolated cell with an affinity reagent that specifically binds the chaperone;
isolating there from any bound target; and
determining the identity of the isolated target.

28. A cyclic peptide composed of from 4 to 30 amino acids, comprising:
a chaperone binding region; and
a target binding region of wholly or partially unknown sequence.

29-32. (canceled)

33. A library of cyclic peptides, each of which comprises a chaperone binding region and a target binding region, wherein the target binding region of each cyclic peptide is unique.

34-36. (canceled)

37. A polynucleotide capable of expressing a cyclic peptide comprising;
a first segment encoding a C-terminal intein domain;
a second segment encoding a linear version of cyclic peptide, said cyclic peptide comprising a chaperone binding region and a target binding region; and
a third segment encoding an N-terminal intein domain, wherein the first, second and third segments are arranged such that the polynucleotide expresses a cyclic peptide.

38-40. (canceled)

41. A library of polynucleotides capable of expressing cyclic peptides, each polynucleotide of the library comprising:
a first segment encoding a C-terminal intein domain;
a second segment encoding a linear version of a cyclic peptide, said cyclic peptide comprising a chaperone binding region and a target binding region; and
a third segment encoding an N-terminal intein domain, wherein said first, second and third segments are arranged such that the ploynucleotide is capable of expressing the cyclic peptide and wherein the target binding region of each expressed cyclic peptide is unique.

42. A host cell comprising a polynucleotide according to claim 41, or progeny thereof.

43-44. (canceled)
